(19) United States
(12) Patent Application Publication
(10) Pub. No.: US 2009/0317429 A1
(43) Pub. Date: Dec. 24, 2009

(54) Aloe vera cotton swab tips

(76) Inventor: Michael Vivian Parris, Kissimmee, FL (US)

Correspondence Address: MICHAEL V. PARRIS, 2175 GRANGER AVE, KISSIMMEE, FL, 34746-2205, US

(21) Appl. No.: 12/214,776

(22) Filed: Jun. 23, 2008

(51) Int. Cl. A61K 36/886; A61K 9/00; A61P 17/00

(52) U.S. Cl. 424/400; 424/744

(57) ABSTRACT

This paper describes how implementing an aloe vera on a Q Tip will enhance the cleaning of the ear. It is a very simple product that the average consumer uses on a daily basis. The only difference is that it will have the available medicinal quality improvement in the product.

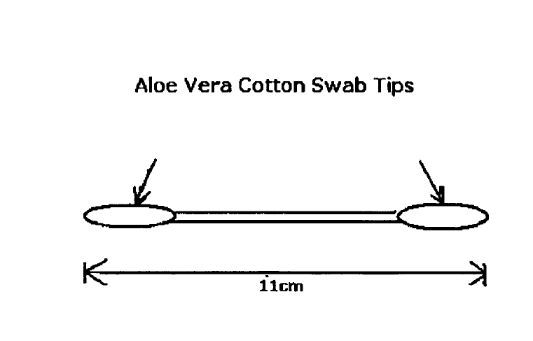

The invention is a regular household Q Tip that has Aloe Vera at both ends. The product is the same length and sized Q Tip that we now use in our homes, as part of our daily cleansing item to clean our ears. Any lay person or persons know what a Q Tip is for and no one has to be skilled in the art to which it pertains or with which it is most nearly connected. Anyone can make the same as specified by 35 U.S.C. 112 Specification—Patent Laws.

My claim is an independent claim based on research of the item there is no known claim by any individual or company. The specific function of this invention is that this product will clean the ear as well as any other product on the market today based on Medical, Traditional and Modern uses.

Medical use—Since the era of ancient Egypt, humans have been using the aloe. Cleopatra was said to have used aloe as a beauty tool. The Egyptians discovered the healing powers of aloe they use it as part of the ingredients of embalming fluid. In the 10^{th} Century the Europeans were introduced to aloe, where it became an importance in many herbal medicines, By the 16^{th} Century, aloe arrived in the West Indies, where it is still grown and harvested. Note—Wikipedia.

Traditional use—There are over 300 different types of aloe, but only a mere few were used traditionally as herbal medicines, this includes aloe perryi found in northeastern Africa and aloe ferox found in South Africa. But the one that tops the list in popularity is aloe vera. It is the most popular and most commonly used. The Greeks and Romans used aloe to treat wounds. Note—Wikipedia

Modern use—Today, the gel found in the leaves is used for soothing minor burns, wounds and various skin conditions like eczema and ringworm. The use of this herbal medicine was popularized in the 1950's in many Western Countries. The gel's effect is nearly immediate, plus it applies a layer over wounds that is said to reduce the chance of infection. Note—Wikipedia.

Benefits from using aloe—Studies that have been conducted states that possible benefits of aloe gel taken internally found improved wound healing in mice, another found a positive effect of lowering the risk factors in patients with heart disease. Some research has shown decreasing fasting blood sugar in diabetic animals given aloe. Note—Wikipedia.

Marketing use—Aloe has been marketed as a remedy for coughs, wounds, ulcers, gastritis, diabetes, cancer, headaches, arthritis, immune-system deficiencies and many other conditions when taken internally. There is, however, evidence of potential adverse side effect for acute hepatitis. Some studies suggest that certain components of aloe, such as, aloe-emodim have genotoxic activity, human clinical traits and rodent carcinogenicity studies do not substantiate a genotoxic risk to humans when aloe products are consumed as directed. Brusick D. Mengs, U 1997—Assessment of the genotoxic risk from laxative senna products.

Chemical properties of Aloe—According to W. A. Shenstone, two classes of aloins are to be recognized: 1. Nataloins which yields picric and oxalic acids with nitric acid and not give a red coloration with nitric acid. 2. Baraloins which yields aloetic acid (C/H2 N3 O5) chrysammic acid (C/H2 N2 O6) picric and oxalic acids with nitric acid being reddened by the acid. The second group may be divided into a-barbaloins, obtained from Barbadocs aloe. Note—Wikipedia.

Species—There are about 400 species of aloes

Aloe arborescens—Used in healthcare

Aloe arisata—Torch Plant Lace Aloe

Aloe dichotoma—quiver tree or kokerboom

Aloe nyeriensis

Aloe varigate—Patridge-breasted aloe, Tiger Aloe

Note—Wikipedia

### SUMMARY

I have personally bought Aloe products from my neighborhood stores. The products have helped me with my health care issues and adding aloe to the end of Q Tips will further enhance the health care of the consumer. This product is safe by looking at the Modern, Traditional and Medical uses. The specifications are clear, exact and concise and will enable lay person to use it safely.

I claim:

1. A Q Tip, comprising of an aloe vera on the cotton swabs, at each end.
